(19) United States
(12) Patent Application Publication
(10) Pub. No.: US 2023/0053297 A1
(43) Pub. Date: Feb. 16, 2023

(54) COMPOSITIONS AND METHODS TO INCREASE SERUM DILEUCINE LEVELS

(71) Applicant: Ingenious Ingredients, LP, Lewisville, TX (US)

(72) Inventors: Ralf Jager, Whitefish Bay, WI (US); Martin Purpura, Spring, TX (US); Shawn D. Wells, Frisco, TX (US); Kylin Liao, Plano, TX (US)

(21) Appl. No.: 17/877,703

(22) Filed: Jul. 29, 2022

Related U.S. Application Data
(60) Provisional application No. 63/226,915, filed on Jul. 29, 2021.

(51) Int. Cl. A23L 33/175

(52) U.S. Cl. CPC A23L 33/175

(57) ABSTRACT

Disclosed herein in is a method of increasing plasma dileucine levels in a subject comprising administering to the subject an effective amount of a composition comprising leucine. In certain embodiments, the composition does not comprise dileucine. In certain embodiments, the amount of increase in plasma dileucine is greater than that resulting from administering a protein supplement comprising a comparable amount of leucine. In certain implementations, administration of the composition results in an increase in plasma dileucine that is from about 10% to about 50% greater than from administering a protein supplement comprising a comparable amount of leucine.

### CROSS-REFERENCE TO RELATED APPLICATION(S)

This application claims the benefit under 35 U.S.C. § 119(e) to U.S. Provisional Application 63/226,915, filed Jul. 29, 2021, and entitled “COMPOSITIONS AND METHODS TO INCREASE SERUM DILEUCINE LEVELS”, which is hereby incorporated herein by reference in its entirety for all purposes.

### BACKGROUND

Dileucine is a dipeptide formed by a peptide bond between two L-Leucines. There is growing recognition of the health benefits associated with enhanced dileucine serum levels. Supplementation with whey protein has been viewed as the most effective method of increasing dileucine levels. However, achieving elevated dileucine levels through nutritional supplementation has been a challenge. Accordingly, there is a need in the art for compositions and methods to more effectively increase dileucine serum levels.

### BRIEF SUMMARY

Disclosed herein in is a method of increasing plasma dileucine levels in a subject comprising administering to the subject an effective amount of a composition comprising leucine. In certain embodiments, the composition does not comprise dileucine.

In certain embodiments, the amount of increase in plasma dileucine is greater than that resulting from administering a protein supplement comprising a comparable amount of leucine. In certain implementations, administration of the composition results in an increase in plasma dileucine that is from about 10% to about 50% greater than from administering a protein supplement comprising a comparable amount of leucine. In further implementations, leucine supplementation results in an increase in plasma dileucine that is about 30% greater than from administering a protein supplement comprising a comparable amount of leucine. In certain embodiments, leucine supplementation results in an increase in plasma dileucine that is about 40% greater than from administering a protein supplement comprising a comparable amount of leucine. In further embodiments, leucine supplementation results in an increase in plasma dileucine that is about 50% greater than from administering a protein supplement comprising a comparable amount of leucine.

According to certain embodiments, following administration the composition, the subject has a peak serum concentration (Cmax) of dileucine of from about 0.3 to about 0.8 μmol/L. In further embodiments, wherein CMax of the subject is from about 0.4 to about 0.8 μmol/L. In yet further embodiments, the CMax of the subject is about 0.6 μmol/L.

According to certain embodiments, following administration the composition, the subject has an Area Under the Curve serum concentration (AUC) of dileucine of from about 25 to about 60 μmol-h/L. In further embodiments, dileucine AUC of the subject is from about 30 to about 50 μmol-h/L. In yet further embodiments, dileucine AUC of the subject is about 40 μmol-h/L.

According to certain embodiments, leucine is administered in an amount of between 500 mg and 8000 mg. In further embodiments, leucine is administered in an amount of between 1000 mg and 5000 mg. In yet further embodiments, the leucine is administered in an amount of between 1500 mg and 3500 mg. In still further embodiments, leucine is administered in an amount of about 2000 mg. In certain embodiments, administration of the composition is repeated daily.

Further disclosed is method of increasing plasma dileucine levels in a subject comprising administering to the subject an effective amount of a composition comprising leucine, wherein the leucine is administered in an amount of between 1500 mg and 3500 mg and wherein leucine supplementation results in plasma CMax dileucine level in the subject that is about 40% greater than in a subject receiving protein supplementation of a comparable dose of leucine.

Further disclosed herein is a method of increasing plasma dileucine levels in a subject comprising administering to the subject an effective amount of a composition comprising leucine, wherein the leucine is administered in an amount of between 1500 mg and 3500 mg and wherein leucine supplementation results in plasma AUC dileucine level that is at least about 30% greater than in a subject receiving supplementation of a comparable dose of plant based dileucine.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWINGS

FIG. 1 is shows individual AUC results by condition.

### DETAILED DESCRIPTION

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

As used herein, the term “subject” refers to the target of administration, e.g. a subject. Thus the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the phrase “identified to be in need of treatment for a disorder,” or the like, refers to selection of a subject based upon need for treatment of the disorder. For example, a subject can be identified as having a need for treatment of a disorder (e.g., a disorder related to excess adipose tissue) based upon an earlier diagnosis by a person of skill and thereafter subjected to treatment for the disorder. It is contemplated that the identification can, in one aspect, be performed by a person different from the person making the diagnosis. It is also contemplated, in a further aspect, that the administration can be performed by one who subsequently performed the administration.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of particles would either completely lack particles, or so nearly completely lack particles that the effect would be the same as if it completely lacked particles. In other words, a composition that is “substantially free of an ingredient or element may still actually contain such item as long as there is no measurable effect thereof.

As used herein, the term “synergistic effect” or grammatical variations thereof means and includes a cooperative action encountered in a combination of two or more active compounds in which the combined activity of the two or more active compounds exceeds the sum of the activity of each active compound alone. The term “synergistically effective amount,” as used herein, means and includes an amount of two or more active compounds that provides a synergistic effect defined above.

Disclosed herein are compositions and methods for increasing plasma dileucine levels in a subject comprising administering to the subject an effective amount of a composition comprising leucine. In certain embodiments, the composition does not comprise dileucine. In certain embodiments, the amount of increase in plasma dileucine is greater than that resulting from administering a protein supplement comprising a comparable amount of leucine. In certain embodiments, leucine supplementation results in an increase in plasma dileucine that is from about 10% to about 50% greater than from administering a protein supplement comprising a comparable amount of leucine. In further embodiments, leucine supplementation results in an increase in plasma dileucine that is about 30% greater than from administering a protein supplement comprising a comparable amount of leucine (e.g., whey protein). Disclosed herein in is a method of increasing plasma dileucine levels in a subject comprising administering to the subject an effective amount of a composition comprising leucine. In certain embodiments, the composition does not comprise dileucine.

According to certain embodiments, following administration the composition, the subject has a peak serum concentration (Cmax) of dileucine of from about 0.3 to about 0.8 μmol/L. In further embodiments, wherein CMax of the subject is from about 0.4 to about 0.8 μmol/L. In yet further embodiments, the CMax of the subject is about 0.6 μmol/L.

According to certain embodiments, following administration the composition, the subject has an Area Under the Curve serum concentration (AUC) of dileucine of from about 25 to about 60 μmol-h/L. In further embodiments, dileucine AUC of the subject is from about 30 to about 50 μmol-h/L. In yet further embodiments, dileucine AUC of the subject is about 40 μmol-h/L.

According to certain embodiments, leucine is administered in an amount of between 500 mg and 8000 mg. In further embodiments, leucine is administered in an amount of between 1000 mg and 5000 mg. In yet further embodiments, the leucine is administered in an amount of between 1500 mg and 3500 mg. In still further embodiments, leucine is administered in an amount of about 2000 mg. In certain embodiments, administration of the composition is repeated daily.

Further disclosed is method of increasing plasma dileucine levels in a subject comprising administering to the subject an effective amount of a composition comprising leucine, wherein the leucine is administered in an amount of between 1500 mg and 3500 mg and wherein leucine supplementation results in plasma CMax dileucine level in the subject that is about 40% greater than from administering a protein supplement comprising a comparable amount of leucine.

Further disclosed herein is a method of increasing plasma dileucine levels in a subject comprising administering to the subject an effective amount of a composition comprising leucine, wherein the leucine is administered in an amount of between 1500 mg and 3500 mg and wherein leucine supplementation results in plasma AUC dileucine level that is at least about 30% greater than in a subject receiving supplementation of a comparable dose of dileucine, or a protein supplement with a comparable dose of leucine (e.g., whey protein).

In further aspects, the disclosed method comprises administering a composition to a subject where the composition comprises dipeptide comprising L-Leucine and one amino acid or amino acid derivative. According to certain embodiments, the amino acid is selected from a group of branched-chain amino acids (BCAA), including, but not limited to, isoleucine, and valine. In further embodiments, the amino acid is selected from the group of essential amino acids, including, but not limited to, histidine, isoleucine, lysine, methionine, phenylalanine, threonine, tryptophan, and valine. In still further embodiments, the amino acid is selected from the group of conditionally essential amino acids including, but not limited to, arginine, cysteine, glutamine, glycine, proline, and tyrosine.

In still further embodiments, the amino acid is selected from the group of non-essential amino acids including, but not limited to, alanine, aspartic acid, asparagine, glutamic acid, serine, selenocysteine and pyrrolsine. In yet further embodiments, the amino acid derivative is selected from the group of creatine, carnitine, creatinol, beta-alanine, taurine, and beta-hydroxy beta-methylbutyrate.

In certain embodiments, leucine is present from about 10% (w/w) to about 90% (w/w). In further embodiments, leucine is present from about 30% to 70% (w/w). In further embodiments, dileucine is present at about 50% (w/w).

In certain aspects of the foregoing embodiments, the composition is substantially free of dileucine.

According to certain further aspects, disclosed is a method for increasing serum dileucine levels of a subject, the method comprising administering to the subject an effective amount of a composition comprising at least about 95% leucine; and between about 0.1%-5% dileucine, and pharmaceutically acceptable salts thereof.

In certain aspects, the compositions administered according to the disclosed methods are produced through bacterial fermentation. According to these embodiments, fermentation techniques are employed utilizing di-/tri-/tetra-peptide-forming enzymes that directly links amino acids, followed by extraction processes.

According to certain embodiments, administration of the disclosed compositions can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a serum dileucine level target, is achieved. For example, when about an increase of about 20% serum dileucine level, about 30% serum dileucine level or more has been achieved. A treatment regimen can include a corrective phase, during which a composition dose sufficient to provide reduction of excess adiposity is administered, followed by a maintenance phase, during which a lower leucine composition dose sufficient maintain to serum dileucine levels is administered.

According to certain embodiments, administration of leucine results in increased levels of plasma dileucine of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to baseline or placebo control.

According to further embodiments, administration of leucine results in increased levels of plasma dileucine of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55%, or at least about 75% (actual % change or median % change) as compared to a subject receiving a comparable dose of dileucine.

In a further aspect, the disclosed compositions increase serum dileucine levels when administered at an oral dose of greater than about 200 mg per day in a human. In a yet further aspect, the disclosed compounds increase serum dileucine levels when administered at an oral dose of greater than about 300 mg per day in a human. In a still further aspect, the disclosed compounds increase serum dileucine levels when administered at an oral dose of greater than about 400 mg per day in a human. In an even further aspect, the disclosed compounds increase serum dileucine levels when administered at an oral dose of greater than about 500 mg per day in a human. In a further aspect, the disclosed compounds increase serum dileucine levels when administered at an oral dose of greater than about 750 mg per day in a human. In a yet further aspect, the disclosed compounds increase serum dileucine levels when administered at an oral dose of greater than about 1000 mg per day in a human. In a still further aspect, the disclosed compounds increase serum dileucine levels when administered at an oral dose of greater than about 2000 mg per day in a human. In an even further aspect, the disclosed compounds increase serum dileucine levels when administered at an oral dose of greater than about 3000 mg per day in a human. In a yet further aspect, the disclosed compounds increase serum dileucine levels when administered at an oral dose of greater than about 5000 mg per day in a human.

In still further embodiments, the disclosed composition is administered to a subject in an amount sufficient to provide a daily dose of leucine of about 1 mg to about 10,000 mg, about 25 mg to about 5000 mg, about 50 mg to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, or about 4100 mg.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

### Food Products

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

### Food Ingredients

Compositions of the present disclosure may take the form of a food ingredient and/or feed ingredient.

As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

### Functional Foods

Compositions of the disclosure may take the form of functional foods.

As used herein, the term “functional food” means food which is capable of providing not only a nutritional effect but is also capable of delivering a further beneficial effect to the consumer.

Accordingly, functional foods are ordinary foods that have components or ingredients (such as those described herein) incorporated into them that impart to the food a specific function—e.g. medical or physiological benefit—other than a purely nutritional effect.

Although there is no legal definition of a functional food, most of the parties with an interest in this area agree that they are foods marketed as having specific health effects beyond basic nutritional effects.

Some functional foods are nutraceuticals. Here, the term “nutraceutical” means a food which is capable of providing not only a nutritional effect and/or a taste satisfaction, but is also capable of delivering a therapeutic (or other beneficial) effect to the consumer. Nutraceuticals cross the traditional dividing lines between foods and medicine.

### Medical Foods

Compositions of the present disclosure may take the form of medical foods.

By “medical food” it is meant a food which is formulated to be consumed or administered with or without the supervision of a physician and which is intended for a specific dietary management or condition for which distinctive nutritional requirements, based on recognized scientific principles, are established by medical evaluation.

### EXAMPLES

The following examples are put forth so as to provide those of ordinary skill in the art with a complete disclosure and description of certain examples of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

Morifuji et al. (J. Agric. Food Chem. 2010, 58, 8788-8797) has shown that whey and soy protein do not contain free dileucine. However, animal protein hydrolysates (whey protein) contain greater amounts of dileucine, compared plant proteins (soy protein). Supplementation with whey protein or whey protein hydrolysate resulted in greater plasma concentrations of dileucine compared to soy protein or soy protein hydrolysate.

In this study we investigated if supplementation with the individual amino acid leucine would change plasma dileucine concentrations and how those potential changes compare to the currently best source to increase plasma dileucine levels, whey protein.

### METHODS

### Experimental Design

Five healthy men between the ages of 18-50 years of age were recruited to participate in this study. Prior to beginning the study, all participants signed an informed consent document and complete a healthy history questionnaire to determine study eligibility.

All participants reported to the laboratory between 06:00-10:00 hours and observing an 8-10 hour fast. For the two days prior to testing, study participants were given a standardized meal plan to help control for changes in dietary intake prior to testing. Upon arrival, participants heart rate, blood pressure, body mass, height and body composition were measured using DEXA. Prior to body composition assessment, study participants were tested for appropriate hydration status. Using standard multiple sample phlebotomy approaches, approximately 10 mL of venous blood was collected from a forearm vein prior to being administrated a single dose of either leucine (2-gram dose) or whey protein isolate (25-gram dose). In a time course fashion, subsequent venous blood samples were collected 30, 60, 90, 120 and 240 minutes after ingestion of their assigned supplement. Participants were provided 250 mL of cold water to ingest after each blood collection. Study participants returned approximately 3-7 days after completion of the previous study visit to complete an identical testing session as previously described while receiving the alternative treatment.

### Results

In accordance with Morifuji et al. (J. Agric. Food Chem. 2010, 58, 8788-8797), whey protein increased plasma dileucine levels, however, unexpected and surprisingly, leucine supplementation increased plasma dileucine levels to an even greater extent, +40.5% compared to whey protein (as AUC).

Subject Demographics
Five healthy males participated in this investigation.

| Variable | Mean | SD | Minimum | Maximum |
|---|---|---|---|---|
| Age (years) | 30.2 | 3.0 | 26 | 34 |
| Height (cm) | 185.1 | 12.5 | 171.5 | 202 |
| Weight (kg) | 93.9 | 7.8 | 85.6 | 103.5 |
| Body Mass Index (kg/m2) | 27.5 | 1.9 | 24.6 | 29.4% |
| Bodyfat (%) | 18.6 | 6.2 | 10.7 | 27.5 |
| Resting Heart Rate (beats/min) | 64.4 | 6.8 | 58 | 75 |
| Systolic Blood Pressure (mm Hg) | 120.2 | 11.0 | 107 | 131 |
| Diastolic Blood Pressure (mm Hg) | 71.6 | 8.5 | 58 | 79 |
| Dileucine Pharmacokinetic | | | | |
| Outcomes 2 grams of leucine | | | | |
| C_{Max} Dileucine (μmol/L) | 0.61 ± 0.43 | | | |
| AUC Dileucine (μmol-h/L) | 37.1 ± 20.2 | | | |
| 25 grams of whey protein isolate | | | | |

| | |
|---|---|
| C_{Max} Dileucine (μmol/L) | 0.26 ± 0.19 |
| AUC Dileucine (μmol-h/L) | 26.4 ± 14.3 |

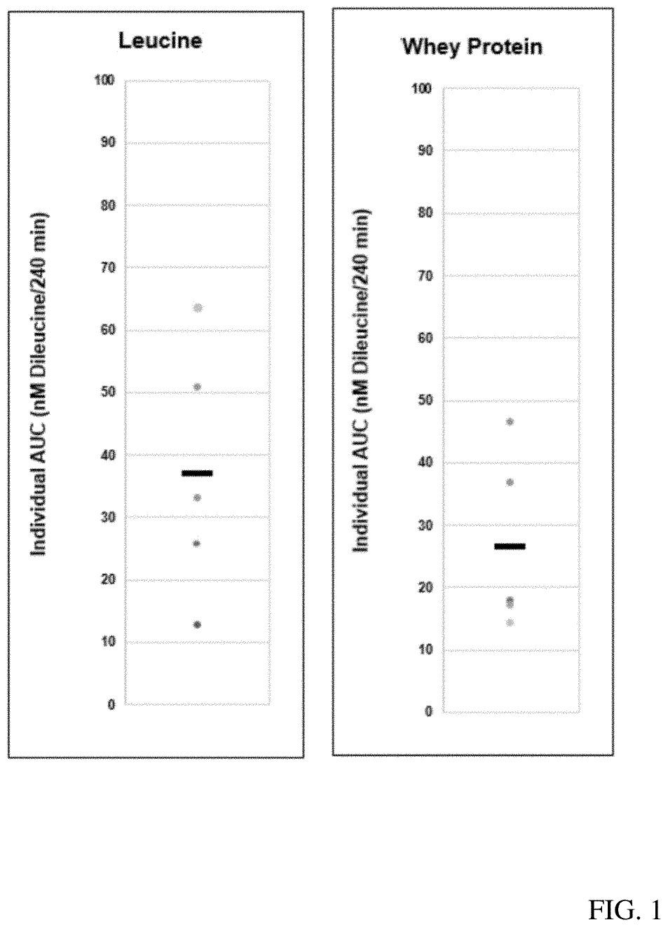

Individual AUC results by condition are shown in FIG. 1.

Although the disclosure has been described with reference to preferred embodiments, persons skilled in the art will recognize that changes may be made in form and detail without departing from the spirit and scope of the disclosed apparatus, systems and methods.

What is claimed is:

1. A method of increasing plasma dileucine levels in a subject comprising administering to the subject an effective amount of a composition comprising leucine.

2. The method of claim 1, wherein the composition does not comprise dileucine.

3. The method of claim 1, wherein the amount of increase in plasma dileucine is greater than that resulting from administration of a protein supplement comprising a comparable amount of leucine.

4. The method of claim 3, wherein administration of the composition results in an increase in plasma dileucine that is from about 10% to about 50% greater than from administering a protein supplement comprising a comparable amount of leucine.

5. The method of claim 4, wherein administration of the composition results in an increase in plasma dileucine that is about 30% greater than from administering a protein supplement comprising a comparable amount of leucine.

6. The method of claim 4, wherein administration of the composition results in an increase in plasma dileucine that is about 40% greater than from administering a protein supplement comprising a comparable amount of leucine.

7. The method of claim 4, wherein administration of the composition results in an increase in plasma dileucine that is about 50% greater than from administering a protein supplement comprising a comparable amount of leucine.

8. The method of claim 1, wherein following administration the composition, the subject has a peak serum concentration (Cₘₐₓ) of dileucine of from about 0.3 to about 0.8 μmol/L.

9. The method of claim 8, wherein C_{Max} of the subject is from about 0.4 to about 0.8 μmol/L.

10. The method of claim 8, wherein C_{Max} of the subject is about 0.6 μmol/L.

11. The method of claim 1, wherein following administration the composition, the subject has an Area Under the Curve serum concentration (AUC) of dileucine of from about 25 to about 60 μmol-h/L.

12. The method of claim 11, wherein dileucine AUC of the subject is from about 30 to about 50 μmol-h/L.

13. The method of claim 12, wherein dileucine AUC of the subject is about 40 μmol-h/L.

14. The method of claim 1, wherein the leucine is administered in an amount of between 500 mg and 8000 mg.

15. The method of claim 14, wherein the leucine is administered in an amount of between 1000 mg and 5000 mg.

16. The method of claim 15, wherein the leucine is administered in an amount of between 1500 mg and 3500 mg.

17. The method of claim 16, wherein the leucine is administered in an amount of about 2000 mg.

18. The method of claim 1, wherein administration of the composition is repeated daily.

19. A method of increasing plasma dileucine levels in a subject comprising administering to the subject an effective amount of a composition comprising leucine, wherein the leucine is administered in an amount of between 1500 mg and 3500 mg and wherein leucine supplementation results in plasma C_{Max} dileucine level in the subject that is about 40% greater than from administering a protein supplement comprising a comparable amount of leucine.

20. A method of increasing plasma dileucine levels in a subject comprising administering to the subject an effective amount of a composition comprising leucine, wherein the leucine is administered in an amount of between 1500 mg and 3500 mg and wherein leucine supplementation results in plasma AUC dileucine level that is at least about 30% greater than from administering a protein supplement comprising a comparable amount of leucine.
